(19) United States
(12) Patent Application Publication
(10) Pub. No.: US 2026/0266835 A1
(43) Pub. Date: Sep. 10, 2026

(54) Method for Simultaneously Enriching Intrinsically Disordered Proteins and Associated Three-Dimensional Genomic Structures Thereof

(71) Applicant: Academy of Military Medical Sciences, Beijing (CN)

(72) Inventors: Zhihu ZHAO, Beijing (CN); Jiabao HOU, Beijing (CN); Yan ZHANG, Beijing (CN); Dejian XIE, Beijing (CN); Wenlong SHEN, Beijing (CN); Ping LI, Beijing (CN); Junwei DENG, Beijing (CN)

(21) Appl. No.: 19/559,594

(22) Filed: Mar. 6, 2026

(30) Foreign Application Priority Data
Mar. 7, 2025 (CN) 202510265135.9

(51) Int. Cl. G01N 33/68; C07K 14/47; C12N 15/10; G01N 1/40

(52) U.S. Cl. CPC G01N 33/6848; C07K 14/47; C12N 15/1093; G01N 1/4044; G01N 1/4077; G01N 2001/4094; G01N 2333/46

(57) ABSTRACT

A method for simultaneously enriching intrinsically disordered proteins (IDPs) and associated three-dimensional genomic structures thereof is provided, belonging to the field of molecular biology technology. The method of the present disclosure includes the steps of: cross-linking cells with formaldehyde and disuccinimidyl glutarate (DSG), then performing enzymatic digestion with restriction endonuclease Dpn II, end repair, and proximity ligation; subjecting nuclei to sonication to obtain a sonicated product after the ligation, dividing the sonicated product into two parts, with one subjected to DNA-protein complexe capture using a biotinylated isoxazole, protein digestion with a protease, DNA extraction and purification, and sequencing library construction from the purified DNA; and the other subjected to nuclease treatment, protein extraction, and protein mass spectrometry analysis. The present disclosure enables genome-wide and non-destructive detection of IDPs and associated three-dimensional chromatin structures, thereby providing an effective approach for their structural analysis.

### CROSS-REFERENCE TO RELATED APPLICATION

This patent application claims the benefit and priority of Chinese Patent Application No. 202510265135.9 filed with the China National Intellectual Property Administration on Mar. 7, 2025, the disclosure of which is incorporated by reference herein in its entirety as part of the present application.

### TECHNICAL FIELD

The present disclosure belongs to the field of molecular biology technology, and specifically relates to a method for simultaneously enriching intrinsically disordered proteins and associated three-dimensional genomic structures thereof.

### BACKGROUND

Intrinsically disordered proteins (IDPs), which account for one third of the human proteome, are characterized by the absence of a stable three-dimensional structure under physiological conditions and play important roles in various biological processes. IDPs possess the ability to form biomolecular condensates through phase separation, a process that promotes the reorganization of three-dimensional chromatin structure, thereby exerting significant effects on gene regulation, development, and disease progression. For example, heterochromatin protein 1 (HP1) mediates phase separation, leading to chromatin compaction and the formation of heterochromatin domains. Similarly, CCCTC-binding factor (CTCF) enhances chromatin A-A compartment interactions, maintains the self--renewal of embryonic stem cells (ESCs), and inhibits their differentiation into neural progenitor cells. In addition, ubiquitously transcribed tetratricopeptide repeat on chromosome X (UTX) is an important tumor suppressor with strong phase-separation ability; when its intrinsically disordered regions (IDRs) are deleted, UTX loses its condensation ability, leading to tumorigenesis.

Currently, there are two main strategies to study the relationship between IDPs and three-dimensional chromatin structures. The Cas9-small guide RNA-biotin-streptavidin droplet-based enrichment (Casl-Drop) system allows the induction of liquid condensates by controlling IDPs at specific genomic loci, thereby enabling the observation of changes in three-dimensional chromatin structures. Another approach involves the use of 1,6-hexanediol to disrupt intracellular phase separation properties, and by treating cells for different duration, the impact of phase separation on changes in three-dimensional chromatin structures can be detected. However, this approach cannot provide insights into the changes in IDPs and three-dimensional chromatin structure under their native intracellular state. Therefore, there remains a lack of a method enabling genome-wide and non-destructive detection of IDPs and associated three-dimensional chromatin structures.

Biotinylated isoxazole (b-isox) is a small molecule that can enrich a broad spectrum of IDPs through the formation of microcrystals, the discovery of which has provided a powerful tool for exploring IDP proteomics. Recent studies have used b-isox to systematically identify potential phase-separating proteins in Arabidopsis thaliana and rice. In addition, in 2023, the DisP-seq method, which combines b-isox precipitation with next-generation sequencing, provided new insights into protein-DNA interactions. This method identified one-dimensional genomic features bound by IDPs, but it failed to characterize the three-dimensional chromatin structural features associated with IDPs.

### SUMMARY

In view of this, it is an objective of the present disclosure to provide a method for simultaneously enriching intrinsically disordered proteins (IDPs) and associated three-dimensional genomic structures thereof, which allows the simultaneous enrichment of the IDPs and the three-dimensional structures they mediate and/or bind.

To achieve the above objective of the present disclosure, the present disclosure provides the following technical solutions.

The present disclosure provides a method for simultaneously enriching IDPs and associated three-dimensional genomic structures thereof, including the steps of: cross-linking and lysing test cells, and extracting nuclei; subjecting the extracted nuclei to enzymatic digestion with restriction endonuclease Dpn II, end repair, and proximity ligation; subjecting the proximity-ligated nuclei to sonication to obtain a sonicated product; dividing the sonicated product into two parts to obtain product I and product II; contacting the product I with a biotinylated isoxazole to enrich sonicated DNA-protein complexes, and subjecting the sonicated DNA-protein complexes to washing, elution, and protease treatment; extracting and purifying DNA after the protease treatment, and constructing a sequencing library from the purified DNA; and treating the product II with a nuclease, extracting proteins, and performing protein mass spectrometry analysis.

In some embodiments, the test cells are cross-linked using formaldehyde and disuccinimidyl glutarate.

In another embodiment, the cross-linking includes the steps of: resuspending the test cells in 1× phosphate-buffered saline (PBS) solution, adding formaldehyde for incubation for 8-15 min, then terminating a reaction by adding glycine; after terminating the reaction, washing resulting cells with 1×PBS solution, and resuspending the washed cells in 1×PBS solution containing 3 mM disuccinimidyl glutarate, incubating the resuspended cells at room temperature for 30-50 min, then adding glycine for continued incubation ; washing the incubated cells with 1×PBS solution containing 0.05% bovine serum albumin, adjusting cell concentration, perfoming centrifugation, discarding a resulting supernatant, and collecting a resulting cell pellet for cryopreservation in liquid nitrogen.

In some embodiments, the lysis includes the steps of: resuspending the cross-linked cells in 1× lysis buffer, then adding a protease inhibitor for incubation on ice for 10-20 min, then performimg centrifugation and discarding a resulting supernatant to obtain precipitate 1; washing the precipitate 1 using 1× NEBuffer 3.1, performing centrifugation and discarding a resulting supernatant to obtain precipitate 2; and resuspending the precipitate 2 in 1× NEBuffer 3.1, adding 1% sodium dodecyl sulfate (SDS) for incubationg for 8-12 min, then terminating the lysis with 10% TRITON X-100.

In some embodiments, a reaction solution for the end repair includes 5-10 μL of 10× NEBuffer 3.1, 1-2 μL of 10 mM 2′-deoxycytidine 5′-triphosphate (dCTP), 1-2 μL of 10 mM 2′-deoxyguanosine 5′-triphosphate (dGTP), 1-2 μL of 10 mM 2′-deoxythymidine 5′-triphosphate (dTTP), 10-20 μL of 1 mM biotin-14- 2′-deoxyadenosine 5′-triphosphate (dATP), and 8-15 μL of 5 U/μL DNA polymerase I large fragment Klenow.

In some embodiments, a reaction solution for the proximity ligation includes 100-150 μL of 10× T4 ligation buffer, 100-150 μL of 10% TRITON X-100, 40-60 μL of T4 DNA ligase and 300-400 μL of MILLI-Q water.

In some embodiments, a reaction solution for the sonication includes a sonication buffer containing 8-15 μL of protease inhibitor; and the sonication buffer includes 40-60 mM 4-(2-hydroxyethyl)-1-piperazineethanesulfonic acid (HEPES), 120-160 mM NaCl, 0.5-2 mM ethylenediaminetetraacetic acid (EDTA), 0.5-2% TRITON X-100 and 0.05-0.2% SDS.

In some embodiments, enrichment using a biotinylated isoxazole includes the steps of: adding 80-120 μM biotinylated isoxazole after the sonication for incubating with rotation at 4°C for 0.5-2 h.

In some embodiments, the washing is performed using a buffer including 1× protease inhibitor, 0.05-0.2 mM phenylmethylsulfonyl fluoride (PMSF), 10-30 mM β-mercaptoethanol, 10-30 mM Tris-HCl, 120-180 mM NaCl, 3-8 mM MgCl₂, 0.1-0.8% NP-40 and 5-15% glycerol.

In some embodiments, the elution buffer includes 5-15 mM Tris-HCl, 0.05-0.2% SDS, 120-180 mM NaCl and 3-8 mM DL-1,4-dithiothreitol (DTT).

Compared with the conventional technology, embodiments of the present disclosure have the following beneficial effects.

The present disclosure discloses a method for simultaneously enriching IDPs and associated three-dimensional genomic structures (DisP-HiC, Disordered protein - High-throughput chromosome conformation capture). In the present disclosure, cells are cross-linked using formaldehyde and disuccinimidyl glutarate (DSG) to fix the three-dimensional structural features of chromatin; and the ligation system and enzymatic digestion system are further optimized to improve ligation efficiency; after sonication of the ligated product, the sonicated product is collected and divided into two parts. One part of the product is used to capture DNA-protein complexes using biotinylated isoxazole, followed by protein digestion with a protease, and DNA library construction for sequencing; the other part of the product is treated with a nuclease, followed by protein extraction, and protein mass spectrometry analysis. The method of the present disclosure may simultaneously enrich IDPs and the three-dimensional structures they mediate and/or bind, enabling genome-wide and non-destructive detection of the three-dimensional chromatin structures bound by IDPs, and thus provides an effective approach for studying the three-dimensional chromatin structural features mediated by IDPs on a genome-wide scale.

### BRIEF DESCRIPTION OF THE DRAWINGS

FIG. 1 is a schematic diagram showing the experimental workflow of the DisP-HiC method of the present disclosure;

FIG. 2 shows the gel electrophoresis analysis on the products after Dpn II digestion and after proximity ligation in the DisP-HiC method of the present disclosure;

FIG. 3 shows the detection of the sonication time that yields the highest DNA concentration after disruption of nuclei with different sonication duration in the DisP-HiC method of the present disclosure;

FIGS. 4A-4B show the data analysis results of the DisP-HiC method of the present disclosure, where FIG. 4A shows the statistical proportions of intra-/inter-chromosomal interaction read pairs and the proportions of interactions with distances greater than/less than 10,000 base pairs in the DisP-HiC data, FIG. 4B shows the correlation of the results from three replicate experiments, and FIG. 4C shows a heatmap plotted based on different intra-chromosomal/inter-chromatin interactions, with 1, 2, 3 ... X representing the chromosome numbers, respectively;

FIG. 5 shows the enrichment of the K562 cell proteome using biotinylated isoxazole in the DisP-HiC method of the present disclosure;

FIGS. 6A-6B show the results in micrococcal nuclease digestion experiments, where FIG. 6A shows an electrophoresis image of 1-minute digestion of cell nuclei with different enzyme amounts, and FIG. 6B shows the results of proximity ligation after digestion;

FIGS. 7A-7C show a data quality control diagram after library construction and sequencing based on micrococcal nuclease digestion, where FIG. 7A shows the statistics of mapped/unmapped reads to the genome and repeated/unrepeated mapped read pairs, FIG. 7B shows the proportions of inter-/intra-chromosomal sequences and insert fragments of different sizes, and FIG. 7C shows a heatmap plotted based on different intra-chromosomal/inter-chromatin interactions after micrococcal nuclease digestion, with 1, 2, 3 ... X representing the chromosome numbers, respectively;

FIGS. 8A-8B show the optimized proximity ligation process based on micrococcal nuclease digestion experiments, where FIG. 8A shows an electrophoresis image of 1-minute digestion of cell nuclei with different enzyme amounts, and FIG. 8B shows an electrophoresis image of the proximity ligation products after treating the digested products with sodium dodecylsulfate.

### DETAILED DESCRIPTION OF THE EMBODIMENTS

The present disclosure provides a method for simultaneously enriching IDPs and associated three-dimensional genomic structures thereof, including the steps of: subjecting test cells to cross-linking and lysis, and extracting nuclei; performing enzymatic digestion with restriction endonuclease Dpn II, end repair, and proximity ligation on the extracted nuclei; subjecting the nuclei upon the proximity ligation to sonication to obtain a sonicated product; dividing the sonicated product into two parts to obtain product I and product II; using a biotinylated isoxazole to enrich the sonicated DNA-protein complexes from the product I, and subjecting the DNA-protein complexes to washing, elution, and protease treatment; extracting and purifying DNA after the protease treatment, and constructing a sequencing library from the purified DNA; and treating the product II with a nuclease, extracting proteins, and performing protein mass spectrometry analysis.

In the present disclosure, preferably, the test cells are cross-linked using formaldehyde and disuccinimidyl glutarate. The specific steps are preferably include: resuspending the test cells in 1×PBS solution, adding formaldehyde for incubation for 8-15 min, then adding glycine to terminate the reaction; after termination of the reaction, performing washing with 1×PBS solution, and resuspending the cells in 1×PBS solution containing 3 mM disuccinimidyl glutarate, incubating at room temperature for 30-50 min, then adding glycine and continuing incubation; performing washing with 1×PBS solution containing 0.05% bovine serum albumin, adjusting the cell concentration, followed by centrifugation, discarding the supernatant, and collecting the cells for cryopreservation in liquid nitrogen. In the present disclosure, the formaldehyde concentration is preferably 35-40% by volume, more preferably 37% by volume. The final concentration of formaldehyde is preferably 0.5-2%, more preferably 1%. The purpose of adding formaldehyde in the present disclosure is to fix the cells. After adding formaldehyde, the incubation is preferably carried out at room temperature for 8-15 min, more preferably for 10 min at room temperature. In the present disclosure, glycine is used to terminate the reaction with formaldehyde. The concentration of the glycine is preferably 2-3 M, more preferably 2.5 M. The final concentration of the glycine is preferably 150-180 mM, more preferably 168 mM. In the present disclosure, the steps for terminating the reaction preferably involve incubating at room temperature for 3-8 min after the addition of glycine, and additionally incubating on ice for 10-20 min.

In the present disclosure, cells are lysed after the cross-linking, where the lysis preferably includes the steps of: resuspending the cross-linked cells in 1× lysis buffer, then adding a protease inhibitor for incubation on ice for 10-20 min, then performimg centrifugation and discarding a resulting supernatant to obtain precipitate 1; washing the precipitate 1 using 1×NEBuffer 3.1, performing centrifugation and discarding a resulting supernatant to obtain precipitate 2; and resuspending the precipitate 2 in 1×NEBuffer 3.1, adding 1% SDS for incubation for 8-12 min, then terminating the reaction with 10% TRITON X-100. In the present disclosure, the lysis buffer preferably includes 10 mM Tris-HCl, pH 8.0, 10 mM NaCl and 0.2% Igepal CA-630. In the present disclosure, sodium dodecylsulfate (SDS) treatment is preferably conducted to make the enzymatic digestion more complete and improve the ligation efficiency.

In the present disclosure, cell nuclei are extracted after cell lysis, the nuclei are subjected to enzymatic digestion, end repair and proximity ligation. The specific steps are preferably as follows. After lysis, preferably 10-15 μL of 10×NEBuffer 3.1 and 400 U of restriction endonuclease Dpn II are added, and mixed gently, followed by digestion overnight on a 37°C shaker; and the resulting mixture is incubated at 60-68°C for 15-25 min to inactivate Dpn II, then cooled on ice for end repair and proximity ligation. In the present disclosure, preferably, a reaction solution for the end repair includes 5-10 μL of 10×NEBuffer 3.1, 1-2 μL of 10 mM dCTP, 1-2 μL of 10 mM dGTP, 1-2 μL of 10 mM dTTP, 10-20 μL of 1 mM biotin-14-dATP, and 8-15 μL of 5 U/μL DNA polymerase I large fragment Klenow; more preferably 6 μL of 10×NEBuffer 3.1, 1.5 μL of 10mM dCTP, 1.5 μL of 10mM dGTP, 1.5 μL of 10mM dTTP, 15 μL of 1mM biotin-14-dATP and 10 μL of 5U/μL DNA polymerase I large fragment Klenow. In some embodiments, a reaction solution for the proximity ligation includes 100-150 μL of 10×T4 ligation buffer, 100-150 μL of 10% TRITON X-100, 40-60 μL of T4 DNA ligase and 300-400 μL of MILLI-Q water; more preferably 120 μL of 10× ligation buffer, 120 μL of 10% TRITON X-100, 50 μL of T4 DNA ligase and 375 μL of MILLI-Q water.

In the present disclosure, cell nuclei are subjected to enzymatic digestion, end repair and proximity ligation treatment followed by sonication, where the specific steps of the sonication are preferably as follows. The cells are washed twice with sonication buffer containing 8-15 μL of protease inhibitor (Thermo Fisher, 78438), and the supernatant is discarded to obtain a precipitate; the precipitate is resuspended in the sonication buffer and transferred to microTUBE-130, and sonication is performed using Covaris M220 sonicator to shear the chromatin. In the present disclosure, the parameters of the Covaris M220 sonicator are preferably set as follows: 75 W, 5% duty cycle, 200 pulses/burst, and for 12 min. In the present disclosure, after sonication, the sample is preferably centrifuged at 12,000 g for 8-15 min, and the supernatant is collected; the supernatant is then brought to a constant volume with sonication buffer.

In the present disclosure, a sonicated product is obtained after sonication, and is then divided into two parts to obtain product I and product II; the sonicated DNA-protein complexes is enriched from the product I using a biotinylated isoxazole, followed by washing, elution, and protease treatment; the DNA is extracted and purified after the protease treatment, and a sequencing library is constructed from the purified DNA. The specific steps are preferably as follows: Biotinylated isoxazole (b-isox) is added to the above constant-volume supernatant to obtain a mixture, which is incubated with rotation at 4°C for 0.5-2 h and then centrifuged at 13,000 g for 10-20 min; the supernatant is discarded and the precipitate is collected; the precipitate is washed twice with wash buffer, then centrifuged at 13,000 g for 10-20 min at 4°C; the supernatant is discarded and the precipitate is collected; the precipitate is resuspended in elution buffer, RNaseA is added, and the mixture is incubated at 37°C with shaking at 600 rpm for 25-35 min. Then, protease K is added, and the mixture is incubated overnight at 60-70°C with shaking at 600 rpm. In the present disclosure, the washing buffer preferably includes 1× protease inhibitor, 0.05-0.2 mM PMSF, 10-30 mM β-mercaptoethanol, 10-30 mM Tris-HCl, 120-180 mM NaCl, 3-8 mM MgCl₂, 0.1-0.8% NP-40 and 5-15% glycerol, more preferably 1× protease inhibitor, 0.1 mM PMSF, 20 mM β-mercaptoethanol, 20 mM Tris-HCl, 150 mM NaCl, 5 mM MgCl₂, 0.5% NP-40 and 10% glycerol. The elution buffer preferably includes 5-15 mM Tris-HCl, 0.05-0.2% SDS, 120-180 mM NaCl and 3-8 mM DTT, more preferably 10 mM Tris-HCl, 0.1%SDS, 150 mM NaCl and 5 mM DTT. All reagents involved in the present disclosure are commercially available through conventional channels.

In the present disclosure, after enrichment of IDPs using biotinylated isoxazole, DNA is extracted and purified, and library construction and sequencing are performed on the purified DNA. The specific steps are preferably as follows: DNA is extracted using 2×VAHTS DNA Clean Beads (N411-02, Vazyme) and eluted with MILLI-Q water according to the manufacturer’s instructions; to remove biotin from unligated DNA ends, the reaction mixture (5 μg DNA, 5 μL of 10×NEBuffer 2.1, 0.125 μL of 10 mM dATP and 0.125 μL of 10mM dGTP and 5 μL of T4 DNA polymerase) is incubated at 18-25°C for 3-4 min; DNA is purified again using VAHTS DNA Clean Beads; finally, a sequencing library is prepared for the Illumina V4 platform using the VAHTS Universal DNA Library Prep Kit (ND610-01, Vazyme), and sequencing is performed on an Illumina platform using TruSeq technology.

In the present disclosure, product II is treated with a nuclease, proteins are extracted, and protein mass spectrometry analysis is performed. The specific steps are preferably as follows: Benzonase nuclease is added to the product II and incubated at 35-37°C for 25-35 min to remove nucleic acids from the system, then the mixture is centrifuged at 13,000 g for 8-15 min, and the supernatant is collected. The supernatant is subjected to protein mass spectrometry analysis. In the present disclosure, the benzonase nuclease is commercially available through conventional channels, and the benzonase nuclease used in the present disclosure is purchased from Merck Millipore, with the catalog number 70664. In the present disclosure, the supernatant to be subjected to protein mass spectrometry analysis is preferably sent to BGI Proteomics for detection and analysis.

The technical solutions provided by the present disclosure will be described in detail in conjunction with the examples below, which cannot be construed as limiting the scope of protection of the present disclosure.

The experimental methods in the following examples are conventional methods unless otherwise specified. The experimental materials used in the following examples are commercially available products unless otherwise specified.

### Example 1

A method for simultaneously enriching IDPs and associated three-dimensional genomic structures thereof is provided, and the specific steps are as follows:

### 1. Cell culture

1.1. The K562 human chronic myeloid leukemia cell line (ATCC, CRL-3344) was cultured in RPMI-1640 complete medium (Thermo Fisher, 31870082). The medium was supplemented with 10% fetal bovine serum (Seradigm Premium Grade HI FBS, VWR), 2 mM l-glutamine (Thermo Fisher, 25030081), and 1× penicillin-streptomycin (Gibco, Life Technologies), and the cells were incubated in a constant-temperature incubator at 37°C with 5% CO₂. For subculture, 3.5 × 10⁵ cells were seeded into 10 mL of complete medium in 10-cm dishes every 2-3 days.

### 2. Cell cross-linking

2.1. Complete medium containing K562 human chronic myeloid leukemia cells was centrifuged at 800 g for 5 min, then the cultured K562 cells were collected. The cells were washed once by complete resuspension at room temperature in 1× PBS.

2.2. The cells were resuspended in 1× PBS at a concentration of 1 × 10⁶ cells/mL;

2.3. To fix the cells, 37% formaldehyde was added to a final concentration of 1%, and the mixture was incubated at room temperature with gentle mixing for 10 min;

2.4. 2.5 M glycine was added to a final concentration of 168 mM to quench the formaldehyde reaction. The mixture was incubated at room temperature for 5 min and then additionally on ice for 15 min;

2.5. The cells were centrifuged at 800 g for 5 min and were washed twice with 1× PBS at room temperature;

2.6. The cells were resuspended in 1× PBS containing 3 mM disuccinimidyl glutarate (DSG) at room temperature and were incubated at room temperature with mixing for 45 min;

2.7. 2.5 M glycine was added to a final concentration of 0.4 M. The mixture was incubated at room temperature for 5 min and then additionally on ice for 15 min;

2.8. The cells were washed twice with 1× PBS (4°C) containing 0.05% bovine serum albumin (BSA);

2.9. The cell concentration was adjusted to 1 × 10⁷ cells/mL with 4°C 1× PBS + 0.05% BSA. The cells were aliquoted into 1.5-mL low-binding Eppendorf EP tubes, were centrifuged at 2,500 g for 5 min, and the supernatant was discarded;

2.10. Cells were flash-frozen in liquid nitrogen and were stored at -80°C.

### 3. Cell lysis and chromatin digestion

3.1. Approximately 1 × 10⁷ cells obtained in step 2 were resuspended in 1 mL of 4°C 1× lysis buffer (10 mM Tris-HCl, pH 8.0; 10 mM NaCl; 0.2% Igepal CA-630), and 10 μL of protease inhibitor (Thermo Fisher, 78444) was added. The mixture was incubated on ice for 15 min and then centrifuged at 2,500 g for 5 min, and the supernatant was discarded;

3.2. Cells were washed twice with 500 μL of 4°C 1× NEBuffer 3.1, and centrifuged at 2,500 g for 5 min, and the supernatant was discarded;

3.3. The precipitate was resuspended in 342 μL of 1× NEBuffer 3.1, 38 μL of 1% SDS was added, and the mixture was incubated at 65°C for 10 min, then the reaction was quenched by addition of 43 μL of 10% TRITON X-100;

3.4. 12 μL of 10× NEBuffer 3.1 and 400 U of Dpn II were added, and after gentle mixing, the mixture was incubated overnight at 37°C with shaking for enzymatic digestion.

### 4. End repair and proximity ligation;

4.1. The digested product obtained in step 3 was incubated at 65°C for 20 min to inactivate Dpn II and was then cooled on ice;

4.2. A reaction solution for end repair was prepared, including 6 μL of 10× NEBuffer 3.1, One and a half microlitters of 10mM dCTP, 1.5 μL of 10mM dGTP, 1.5 μL of 10mM dTTP, 15 μL of 1mM biotin-14-dATP and 10 μL of 5U/μL DNA polymerase I large fragment Klenow;

4.3. Sixty microlitters of the reaction solution for end repair was added to the digested nuclei, then the mixture was incubated at 23°C for 4 h. After the mixture was cooled, a reaction solution for proximity ligation (120 μL of 10× T4 ligation buffer, 120 μL of 10% TRITON X-100, 50 μL of T4 DNA ligase, and 375 μL of Milli-Q water) was added, and the resulting mixture was incubated at 16°C for 4 h.

### 5. Nuclear sonication and enrichment with biotinylated isoxazole

5.1. The product after proximity ligation from step 4 was centrifuged at 2,800 g for 10 min, and the supernatant was discarded. The resultant was washed twice with 1 mL of sonication buffer (50 mM HEPES, pH 7.8; 140 mM NaCl; 1 mM EDTA; 1% TRITON X-100; 0.1% SDS) containing 10 μL of protease inhibitor (Thermo Fisher, 78438), and the supernatant was discarded to obtain a precipitate;

5.2. The precipitate was resuspended in 130 μL of sonication buffer and was transferred to microTUBE-130;

5.3. Chromatin shearing was performed using Covaris M220 sonicator (75 W, 5% duty cycle, 200 pulses/burst, for 12 min);

5.4. The sample was centrifuged at 12,000 g for 10 min, and the supernatant was collected and adjusted to a volume of 600 μL with sonication buffer;

5.5. The 600 μL supernatant obtained in step 5.4 was divided into two equal parts to obtain product I and product II. Then, 100 μM biotinylated isoxazole was added to product I, and the mixture was incubated with rotation at 4°C for 1 h;

5.6. The mixture was centrifuged at 13,000 g for 15 min at 4°C, and the supernatant was discarded to collect the precipitate. The precipitate was washed twice with wash buffer and was centrifuged at 13,000 g for 15 min at 4°C, and the supernatant was discarded to collect the precipitate. The wash buffer has components of 20mM Tris-HCl, pH 7.4; 150 mM NaCl; 5 mM MgCl₂; 0.5% NP-40; 10% glycerol plus 1× protease inhibitor, 0.1 mM PMSF and 20 mM β-mercaptoethanol;

5.7. The precipitate collected in step 5.6 was resuspended in 200 μL of elution buffer (10 mM Tris-HCl, pH 8.0; 0.1% SDS; 150 mM NaCl; 5mM DTT), then 1 μL of RNaseA was added, and the mixture was incubated at 37°C with shaking at 600 rpm for 30 min. Then, 10 μL of protease K was added, and the mixture was incubated overnight at 65°C with shaking at 600 rpm.

### 6. DNA purification and library preparation

6.1. DNA was extracted using 2×VAHTS DNA Clean Beads (N411-02, Vazyme) and eluted with 30 μL of Milli-Q water according to the manufacturer’s instructions;

6.2. To remove biotin from unligated DNA ends, the reaction mixture (5 μg DNA, 5 μL of 10×NEBuffer 2.1, 0.125 μL of 10 mM dATP, 0.125 μL of 10 mM dGTP, 5 μL of T4 DNA polymerase (3000 U/ml, M0203L, NEB)) was incubated at 20°C for 4 h;

6.3. DNA was purified again using VAHTS DNA Clean Beads;

6.4. Finally, a sequencing library was prepared for the Illumina V4 platform using the VAHTS Universal DNA Library Prep Kit (ND610-01, Vazyme), and sequencing was performed on an Illumina platform using TruSeq technology.

### 7. Protein mass spectrometry analysis

1 U/mL of benzonase nuclease (Millipore, catalog number: 70664) was added to the product II and incubated at 37°C for 30 min to remove nucleic acids from the system, then the mixture was centrifuged at 13,000 g for 10 min, and the supernatant was collected. The supernatant was sent to BGI Proteomics for protein mass spectrometry detection.

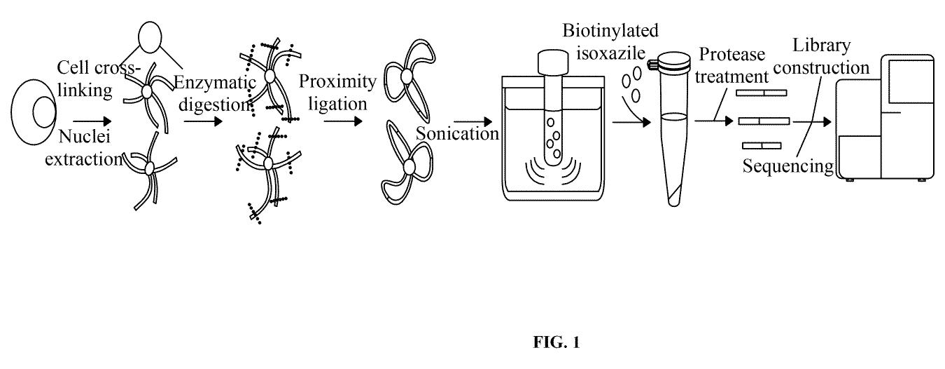

The flow chart of the present disclosure is shown in FIG. 1.

### Comparative Example 1

The preceding steps were performed by following the procedures described in steps 1 and 2 of Example 1.

Differences from Example 1 are as follows.

3.1. Approximately 1 × 10⁷ cells obtained in step 2 were resuspended in 1 mL of 4°C 1× lysis buffer (10 mM Tris-HCl, pH 8.0; 10 mM NaCl; 0.2% Igepal CA-630) and 10 μL protease inhibitor (Thermo Fisher, 78444) was added, followed by 15 min of incubation on ice and a centrifugation at 10000 g for 5 min and the supernatant was discarded;

3.2. Cells were resuspended again in 100 μL of 1× lysis buffer. Next, 0, 1, 5, 10, and 20 U of micrococcal nucleases were added to separate aliquots of the cells, respectively. Digestion was carried out at 37°C for 1 min, then the samples were immediately placed on ice;

3.3. From each of the above five samples, 10 μL of the product was taken, 5 μL of protease K and 35 μL of 1× PBS buffer were added, and the mixture was incubated at 65°C for 30 min before gel electrophoresis was performed.

The remaining 90 μL of sample in step 3.3 was treated at 65°C for 10 min, and centrifuged at 12,000 g for 10 min, and the supernatant was discarded;

3.4. The cells were resuspended in 100 μL of an end repair system (10 μL of 10×NEBuffer 2.1, 2 μL of 100 mM ATP, 5 μL of 100 mM DTT, 68 μL of water, 5 μL of 10 U/µL T4 PNK), and the mixture was incubated at 37°C for 15 min;

3.5. Subsequently, 10 µL of 5 U/µL DNA polymerase large fragment Klenow was added to the product from step 3.4, and the mixture was incubated at 37°C for 15 min;

3.6. An end-labeling system (10 µL of 1 mM Biotin-dATP, 10 µL of 1 mM Biotin-dCTP, with 1 µL of 10 mM dTTP+dGTP, 5 µL of 10× T4 DNA ligase buffer and 24 µL of water added separately) was added to the system from step 3.5. The mixture was incubated at 25°C for 45 min and then at 65°C for 30 min;

3.7. The product was centrifuged at 12,000 g for 10 min, the supernatant was discarded, and the resultant was washed once with 1× PBS;

3.8. A proximity ligation system (425 µL of water, 50 µL of 10× T4 DNA ligase buffer and 25 µL of 400 U/µL T4 DNA ligase) was added for ligation at 25°C for 3 h;

3.9. Ten microlitters of the product was taken separately, 5 µL of protease K and 35 µL of 1× PBS buffer were added, and the mixture was incubated at 65°C for 30 min before gel electrophoresis was performed.

### Comparative Example 2

The preceding steps were performed by following the procedures described in steps 1 and 2 of Example 1.

Differences from Example 1 are as follows.

3.1. Approximately 1 × 10⁷ cells obtained in step 2 were resuspended in 1 mL of 4°C 1× lysis buffer (10 mM Tris-HCl, pH 8.0; 10 mM NaCl; 0.2% Igepal CA-630) and 10 μL protease inhibitor (Thermo Fisher, 78444) was added, followed by 15 min of incubation on ice, and a centrifugation at 10000 g for 5 min, and the supernatant was discarded;

3.2. Cells were resuspended again in 100 μL of 1× lysis buffer. 0, 1, 5, 10, and 20 U of micrococcal nucleases were added to separate aliquots of the cells, respectively. Digestion was carried out at 37°C for 1 min, then the samples were immediately placed on ice;

3.3. From each of the above five samples, 10 μL of the product was taken, 5 μL of protease K and 35 μL of 1× PBS buffer were added, and the mixture was incubated at 65°C for 30 min before gel electrophoresis was performed.

The remaining 90 μL of sample in step 3.3 was treated at 65°C for 10 min, was centrifuged at 12,000 g for 10 min, and the supernatant was discarded;

3.4. The cells were resuspended in 100 μL of an end repair system (10 μL of 10×NEBuffer 2.1, 2 μL of 100 mM ATP, 5 μL of 100 mM DTT, 68 μL of water, 5 μL of 10 U/µL T4 PNK), and the mixture was incubated at 37°C for 15 min;

3.5. Subsequently, 10 µL of 5 U/µL DNA polymerase large fragment Klenow was added to the product from step 3.4, and the mixture was incubated at 37°C for 15 min;

3.6. An end-labeling system (10 µL of 1 mM Biotin-dATP, 10 µL of 1 mM Biotin-dCTP, with 1 µL of 10 mM dTTP + dGTP, 5 µL of 10× T4 DNA ligase buffer and 24 µL of water added separately) was added to the system from step 3.5. The mixture was incubated at 25°C for 45 min and then at 65°C for 30 min;

3.7. Fifty microlitters of 1% SDS was added to the product for resuspending, and the mixture was treated at 65°C for 10 min. The sample was then centrifuged at 12,000 g for 10 min, and the resultant was washed once with 1× PBS;

3.8. A proximity ligation system (425 µL of water, 50 µL of 10× T4 DNA ligase buffer and 25 µL of 400 U/µL T4 DNA ligase) was added for ligation at 25°C for 3 h;

3.9. Ten microlitters of the product was taken separately, 5 µL of protease K and 35 µL of 1× PBS buffer were added, and the mixture was incubated at 65°C for 30 min before gel electrophoresis was performed.

### Result analysis

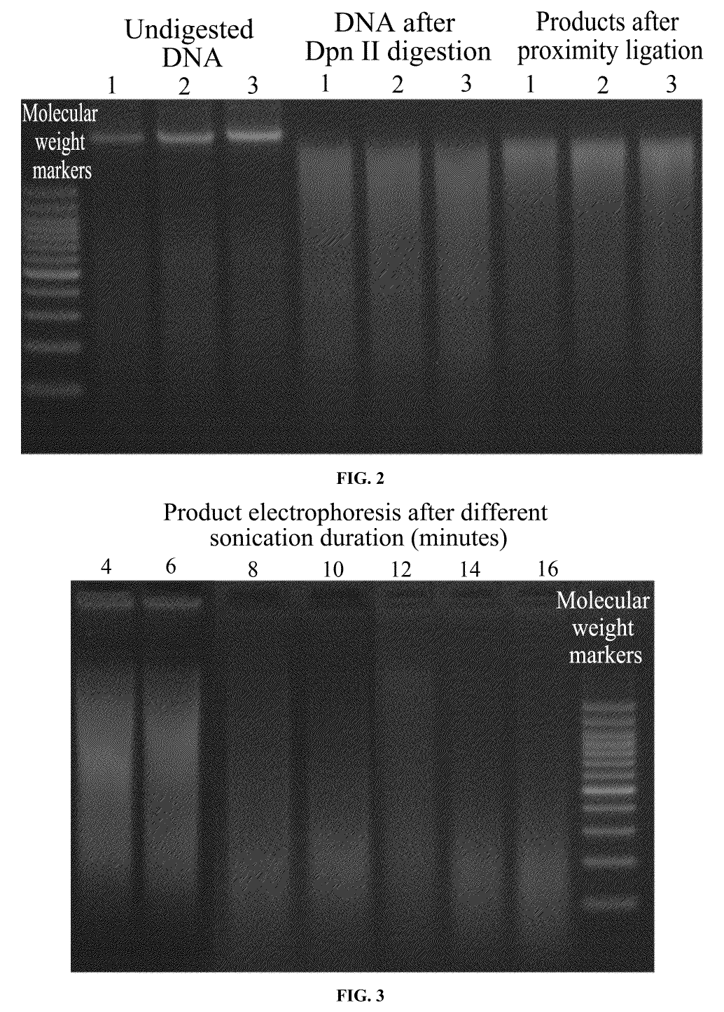

From FIG. 2, it is found that the undigested DNA shows a single, high-integrity band, indicating that the DNA is not degraded; the DNA after DpnII digestion appears as a smear, indicating that digestion is complete; and the products after proximity ligation are larger than those after digestion, indicating that ligation was complete and efficient.

As shown in FIG. 3, after the nuclei is disrupted using different sonication duration, the DNA bands exhibit different sizes. Among these, the library concentration is higher when sonication is performed for 12 min, indicating that this condition is suitable for subsequent sequencing analysis.

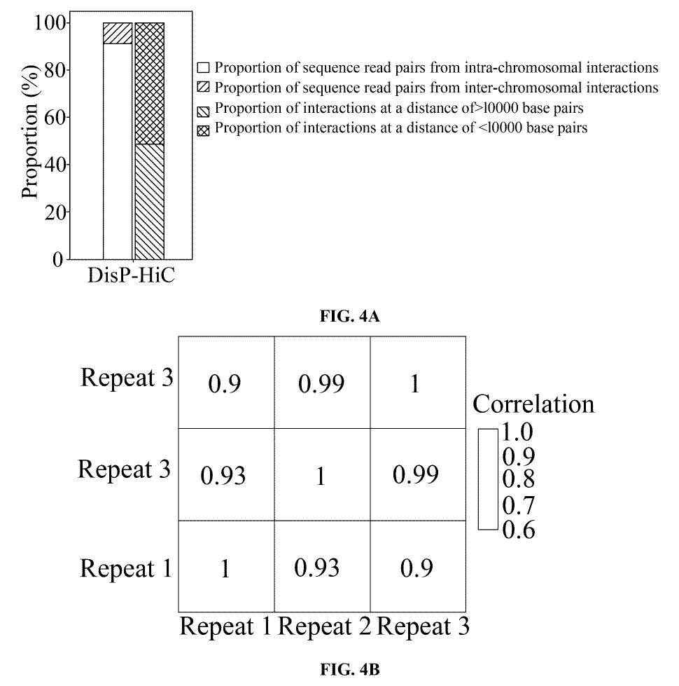

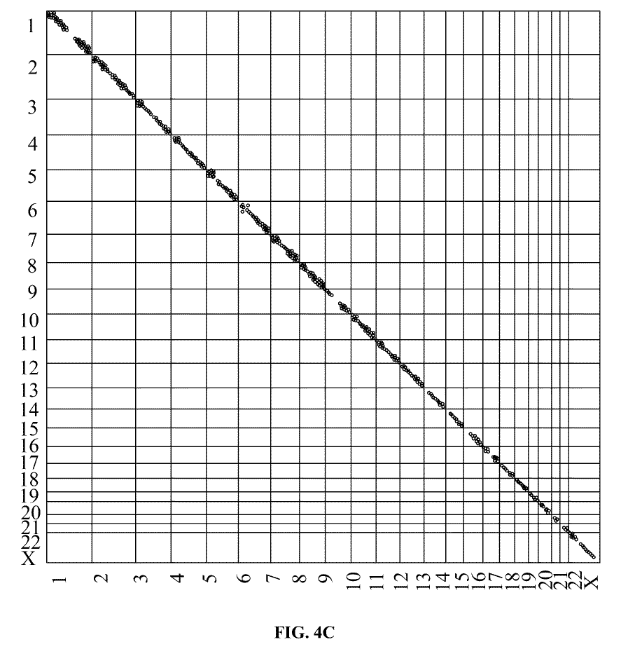

As shown in FIG. 4A, intra-chromatin interactions accounts for 90%, which is much higher than inter-chromatin interactions, and the proportion of long-range intra-chromatin interactions (> 10 kb) is approximately 50%; these results indicate that the library meet the quality control standards for chromatin higher-order structure. FIG. 4B shows that the correlation between data from three replicate experiments using the DisP-HiC method are high, indicating that the method has good reproducibility and technical stability. FIG. 4C shows visual analysis of the data. The background was low and the resolution was high, indicating that the technology had been successfully established and could be used for subsequent analyses.

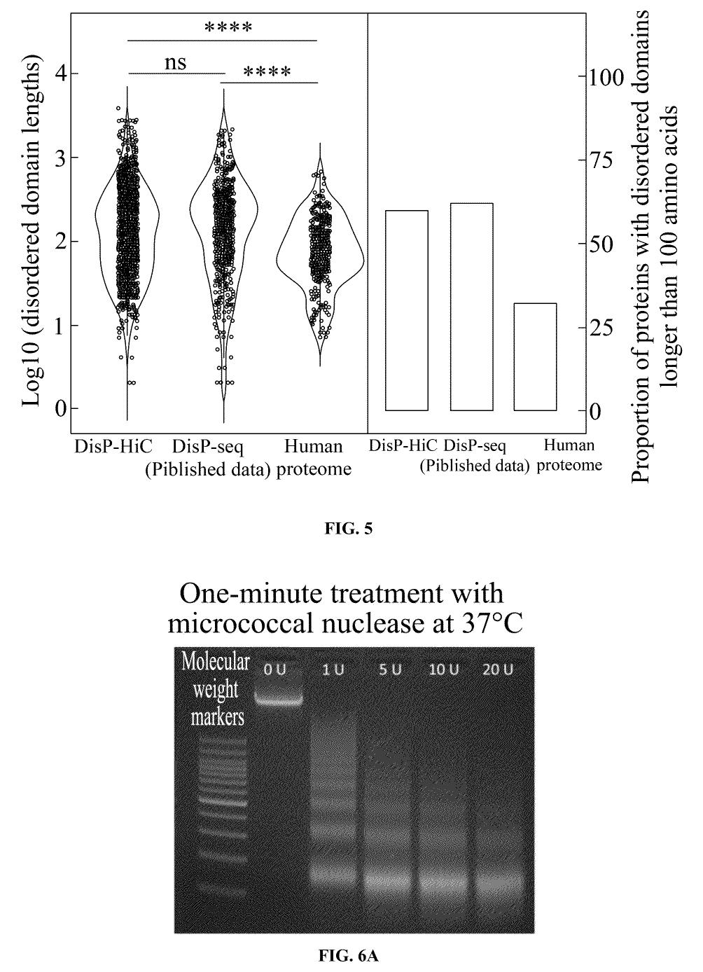

FIG. 5 demonstrates that the yield of intrinsically disordered domains (IDDs) in the K562 proteome and the proportion of proteins containing >100-amino-acid IDDs are significantly higher than those in the random data of the human proteome, with published DisP-seq proteome data used as a reference (from: DisP-seq reveals the genome-wide functional organization of DNA-associated disordered proteins). These results indicate that the method of the present disclosure could effectively enrich IDPs in K562 using biotinylated isoxazole.

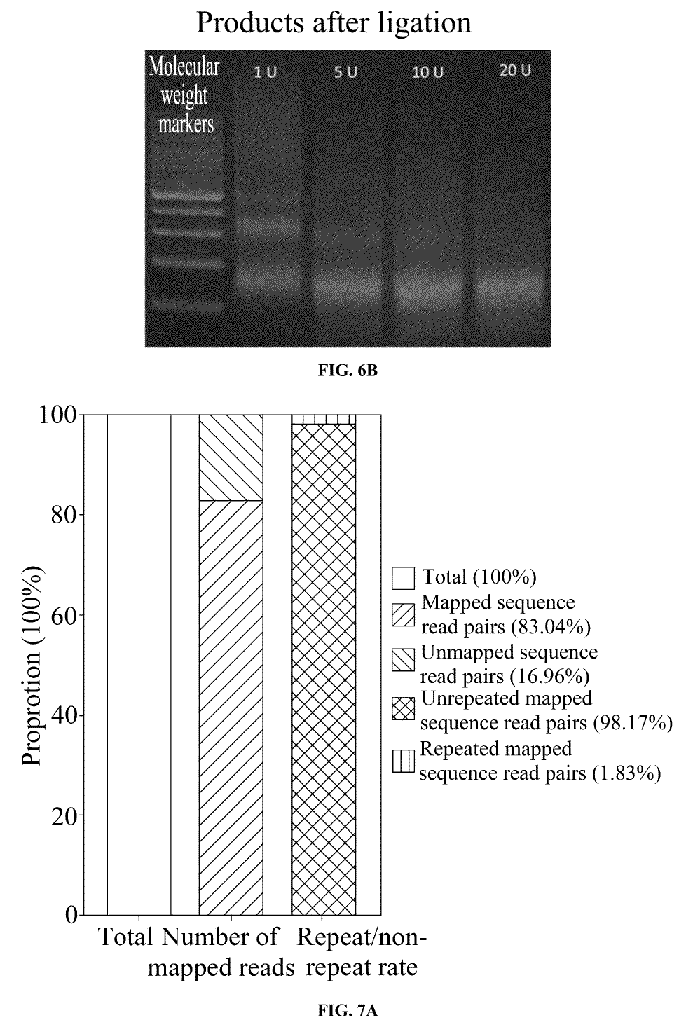

As shown in FIG. 6A, as the amount of micrococcal nuclease increases, the DNA digestion fragments become progressively smaller; and FIG. 6B shows that although proximity ligation was performed on the digested DNA, no obvious increase in DNA length is observed, indicating that the proximity ligation efficiency in this workflow is low.

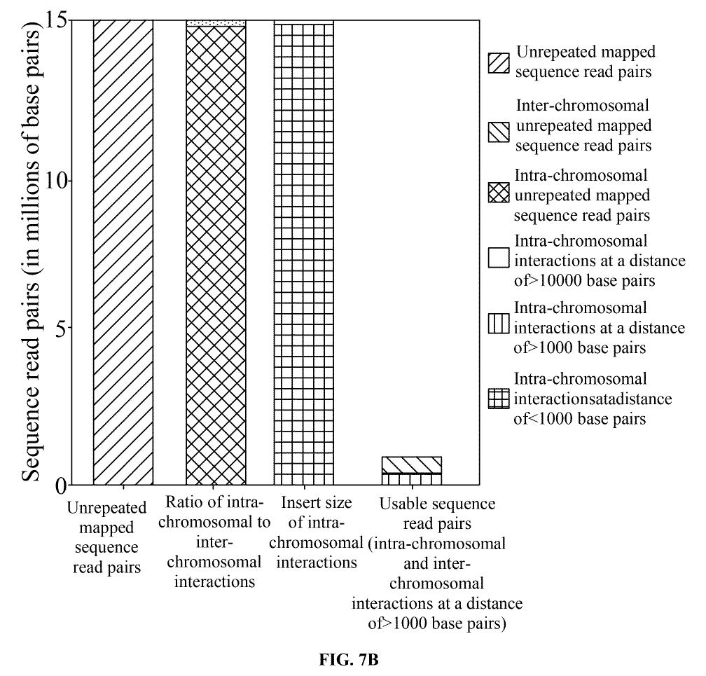

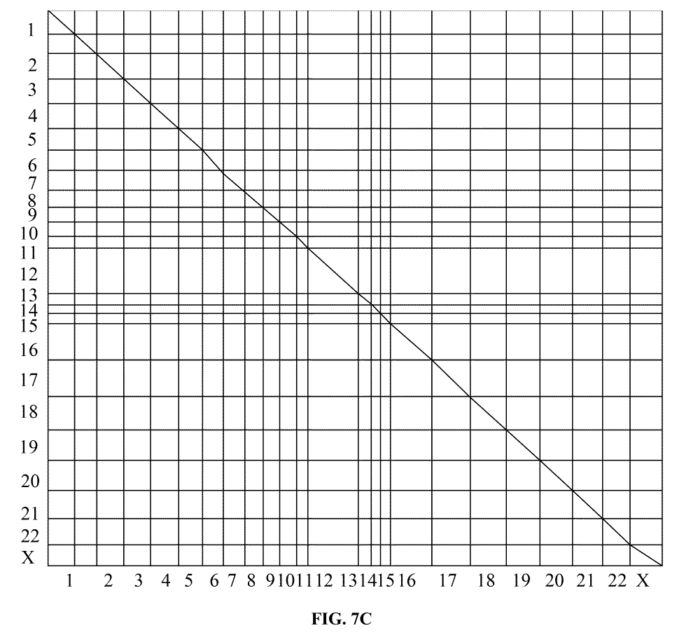

FIGS. 7A-7C show the data generated by the workflow of Comparative Example 1. FIG. 7A indicates that a high proportion of the sequencing read pairs are mapped to the human hg19 reference genome, and the proportion of repeated read pairs in the library is low. FIG. 7B shows that among the read pairs mapped to the reference genome, the proportion of intra-chromosomal interactions is higher than that of inter-chromosomal interactions, but the number of read pairs corresponding to long-range interactions is relatively low, indicating that the proximity ligation efficiency in the experiment is insufficient. FIG. 7C provides a visualization of the data, which exhibit high background noise and low resolution, making it unsuitable for subsequent analysis.

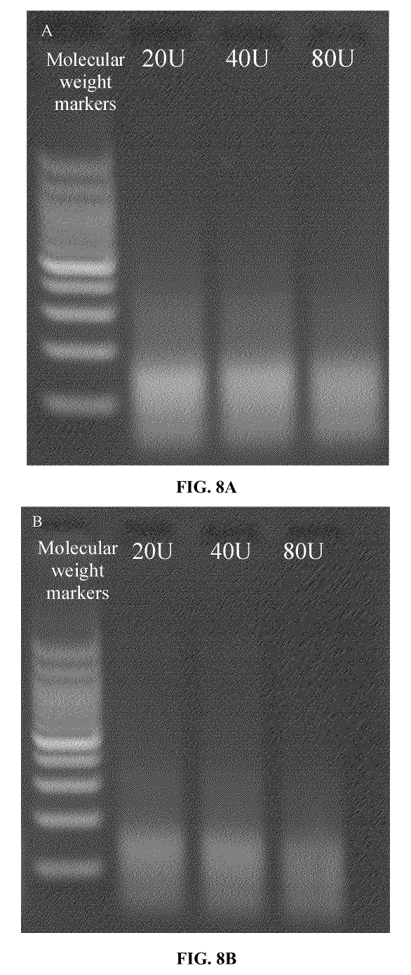

FIGS. 8A-8B show the data generated by the workflow of Comparative Example 2. FIG. 8A demonstrates that micrococcal nuclease has digested the DNA efficiently, and the DNA fragments are significantly reduced in size. FIG. 8B shows that the DNA products remain unchanged after proximity ligation, indicating that the proximity ligation efficiency is low.

The descriptions above are merely the preferred embodiments of the present disclosure. It should be noted that several improvements and modifications may also be made by those of ordinary skilled in the art without departing from the principle of the present disclosure, and these improvements and modifications shall also be considered within the scope of protection of the present disclosure.

What is claimed is:

1. A method for simultaneously enriching intrinsically disordered proteins (IDPs) and associated three-dimensional genomic structures thereof, comprising the steps of: cross-linking and lysing test cells, and extracting nuclei; subjecting the extracted nuclei to enzymatic digestion with restriction endonuclease Dpn II, end repair, and proximity ligation; subjecting the proximity-ligated nuclei to sonication to obtain a sonicated product; dividing the sonicated product into two parts to obtain product I and product II; enriching sonicated DNA-protein complexes from the product I with a biotinylated isoxazole, and subjecting the sonicated DNA-protein complexes to washing, elution, and protease treatment; extracting and purifying DNA after the protease treatment, and constructing a sequencing library from the purified DNA; and treating the product II with a nuclease, extracting proteins, and performing protein mass spectrometry analysis;wherein the test cells are cross-linked using formaldehyde and disuccinimidyl glutarate;
wherein the cross-linking comprises the steps of: resuspending the test cells in 1×phosphate-buffered saline (PBS) solution, adding formaldehyde for incubation for 8-15 min, then terminating a reaction by adding glycine; after terminating the reaction, washing resulting cells with 1×PBS solution, and resuspending the washed cells in 1×PBS solution containing 3 mM disuccinimidyl glutarate, incubating re resuspended cells at room temperature for 30-50 min, then adding glycine for continued incubation; washing the incubated cells with 1×PBS solution containing 0.05% bovine serum albumin, adjusting cell concentration, performing centrifugation, discarding a resulting supernatant, and collecting a resulting cell pellet for cryopreservation in liquid nitrogen;
wherein the enzymatic digestion comprises the steps of adding and mixing 10-15 μL 10× NEBuffer 3.1 and 400 U restriction endonuclease Dpn II following cell lysis, digesting a resulting mixture on a 37 °C shaker overnight, followed by incubation at 60-68 °C for 15-25 min to inactivate the restriction endonuclease Dpn II, cooling on ice, and performing the end repair, and the proximity ligation;
wherein a reaction solution for the end repair comprises 5-10 μL of 10×NEBuffer 3.1, 1-2 μL of 10 mM 2′-deoxycytidine 5′-triphosphate (dCTP), 1-2 μL of 10 mM 2′-deoxyguanosine 5′-triphosphate (dGTP), 1-2 μL of 10 mM 2′-deoxythymidine 5′-triphosphate (dTTP), 10-20 μL of 1 mM biotin-14- 2′-deoxyadenosine 5′-triphosphate (dATP), and 8-15 μL of 5 U/μL DNA polymerase I large fragment Klenow;
wherein a reaction solution for the proximity ligation comprises 100-150 μL of 10×T4 ligation buffer, 100-150 μL of 10% TRITON X-100, 40-60 μL of T4 DNA ligase and 300-400 μL of MILLI-Q water.

2. The method according to claim 1, wherein the lysis comprises the steps of: resuspending the cross-linked cells in 1× lysis buffer, then adding a protease inhibitor for incubation on ice for 10-20 min, then performimg centrifugatoin and discarding a resulting supernatant to obtain precipitate 1; washing the precipitate 1 using 1×NEBuffer 3.1, performing centrifugation and discarding a resulting supernatant to obtain precipitate 2; and resuspending the precipitate 2 in 1×NEBuffer 3.1, adding 1% sodium dodecyl sulfate (SDS) for incubation for 8-12 min, then terminating the lysis with 10% TRITON X-100.

3. The method according to claim 1, wherein a reaction solution for the sonication comprises a sonication buffer containing 8-15 μL of protease inhibitor; and the sonication buffer comprises 40-60 mM 4-(2-hydroxyethyl)-1-piperazineethanesulfonic acid (HEPES), 120-160 mM NaCl, 0.5-2 mM ethylenediaminetetraacetic acid (EDTA), 0.5-2% TRITON X-100 and 0.05-0.2% SDS.

4. The method according to claim 1, wherein enrichment using the biotinylated isoxazole comprises the steps of: adding 80-120 μM biotinylated isoxazole after the sonication for incubationg with rotation at 4°C for 0.5-2 h.

5. The method according to claim 1, wherein the washing is performed using a buffer comprising 1× protease inhibitor, 0.05-0.2 mM phenylmethylsulfonyl fluoride (PMSF), 10-30 mM β-mercaptoethanol, 10-30 mM Tris-HCl, 120-180 mM NaCl, 3-8 mM MgCl₂1-00..8% NP-40 and 5-15% glycerol.

6. The method according to claim 1, wherein the elution is performed in a buffer comprising 5-15 mM Tris-HCl, 0.05-0.2% SDS, 120-180 mM NaCl and 3-8 mM DL-1,4-dithiothreitol (DTT).
